(19) United States
(12) Patent Application Publication
(10) Pub. No.: US 2009/0082759 A1
(43) Pub. Date: Mar. 26, 2009

(54) LASER APPARATUS FOR THE REDUCTION OF PAIN AND INFLAMMATION CAUSED BY INJECTIONS

(75) Inventors: Brian Pryor, Ewing, NJ (US); Sean Xiaolu Wang, Wilmington, DE (US)

Correspondence Address: FRANK F. TIAN, 331-4A THIRD AVENUE, LONG BEACH, NJ, 07740, US

(73) Assignee: BWT PROPERTY, INC., Newark, DE (US)

(21) Appl. No.: 12/237,184

(22) Filed: Sep. 24, 2008

Related U.S. Application Data
(60) Provisional application No. 60/974,987, filed on Sep. 25, 2007.

(51) Int. Cl. A61B 18/20

(52) U.S. Cl. 606/13

(57) ABSTRACT

This invention discloses a laser apparatus to be used pre, during, and/or post medical injections for the reduction of pain caused by the injection. Applications include cosmetic injections such as Botox or dermal filling, mesotherapy, immunization shots, etc. The laser apparatus may comprise a wand or pad/bandage through which laser treatment can be applied to the injection area.

### REFERENCE TO RELATED APPLICATION

This application claims an invention which was disclosed in Provisional Patent Application No. 60/974,987, filed Sep. 25, 2007, entitled “Laser Apparatus for the Reduction of Pain and Inflammation Caused by Injections”. The benefit under 35 USC §119(e) of the above mentioned United States Provisional applications is hereby claimed, and the aforementioned application is hereby incorporated herein by reference.

### FIELD OF THE INVENTION

This invention generally relates to a laser apparatus, and more specifically to a laser apparatus for the reduction of pain and inflammation caused by injections.

### BACKGROUND

Phototherapy or laser therapy has proved to be very efficient in relieving pain and improving wound healing as demonstrated by Wong in U.S. Pat. No. 5,640,978, entitled “Method for pain relief using low power laser light”, by Friedman in U.S. Pat. No. 6,450,170, entitled “Treatment of migraine, post-traumatic headache, tension-type headaches, atypical facial pain, cervical pain and muscle spasm”, and by Masotti et al. in U.S. Pat. No. 6,527,797, entitled “Laser device for treatment of painful symptomatologies and associated method”, as well as numerous related publications. However, none of these references or publication discloses the application of laser therapy for the reduction of pain and inflammation caused by medical or cosmetic injections, where the laser therapy not only treats but also prevents pain and inflammation symptoms.

Therefore, there is a need for using a laser device for the reduction of pain and inflammation caused by medical/cosmetic injections as well as other procedures, where the laser therapy not only treats but also prevents pain and inflammation symptoms.

### SUMMARY OF THE INVENTION

The present invention discloses a laser apparatus to be used pre, during, and/or post medical injections for the reduction of pain caused by the injection. Applications include cosmetic injections such as Botox or dermal filling, mesotherapy, immunization shots, etc. The laser apparatus may comprise a wand or pad/bandage through which laser treatment can be applied to the injection area.

A method for reducing pain and inflammation of a biological tissue caused by medical or cosmetic injections is provided. The method comprises the steps of: providing a light source to produce a light beam; and causing the light beam to interact with the biological tissue before, during or after an injection process to reduce pain and inflammation of the tissue.

An apparatus for reducing pain and inflammation of a biological tissue caused by medical or cosmetic injections is provided. The apparatus comprises a light source to produce a light beam. The light beam interacts with the biological tissue before, during or after an injection process to reduce pain and inflammation of the tissue.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates a first exemplary embodiment of the laser apparatus for the reduction of pain and inflammation caused by injections in accordance with the present invention.

FIG. 2 illustrates a slight variation of the first exemplary embodiment.

FIG. 3 illustrates a second exemplary embodiment of the laser apparatus for the reduction of pain and inflammation caused by injections in accordance with the present invention.

FIG. 4 is an exemplary flow chart in accordance with some embodiments of the present invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to a laser apparatus for the reduction of pain and inflammation caused by injections. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms, such as first and second, top and bottom, and the like, may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

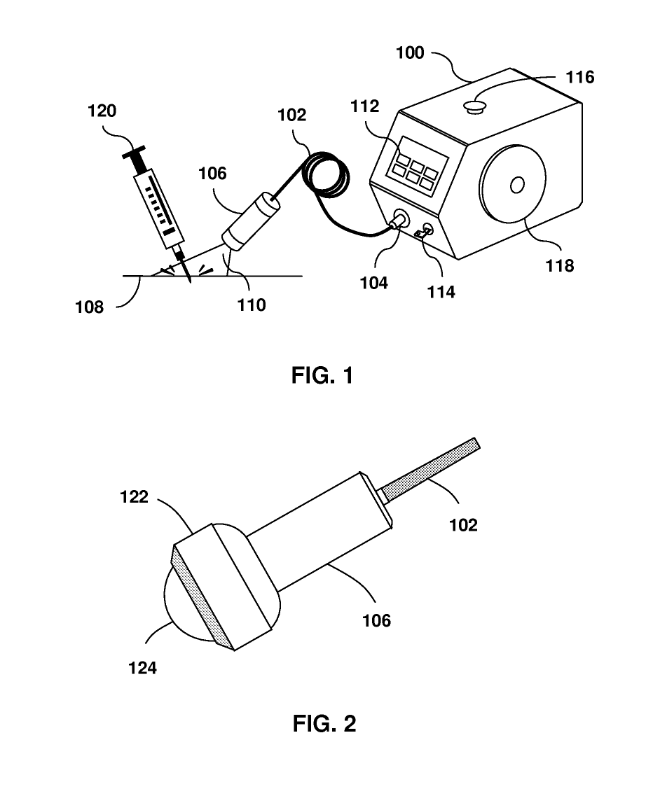

FIG. 1 illustrates one exemplary embodiment of the laser apparatus for the reduction of pain and inflammation caused by Botox (Botulinum toxin) injections. Botox injection is a popular non-surgical injection that temporarily reduces or eliminates frown lines, forehead creases, crows-feet near the eyes and thick bands in the neck. The toxin blocks the nerve impulses and temporarily paralyzes the muscles that cause wrinkles to give the skin a smoother appearance. In FIG. 1, a laser apparatus 100 is utilized to relieve the pain and reduce the inflammation caused by Botox injection. The laser apparatus 100 comprises one or more laser sources (not shown) that produce laser emission in the wavelength range from ultraviolet/visible to infrared. In this specific example, the laser source is a diode laser emitting at 980 nm. The laser emission is coupled into the proximal end of an optical fiber 102 at the output port 104 of the laser apparatus 100. An optical wand 106 at the distal end of the optical fiber 102 is used to output the laser emission to the skin tissue 108 to be treated with Botox injection using a syringe 120. The optical wand 106 comprises optical components (not shown) to control the divergence angle and spot size of the output laser beam 110 so that the intensity of the laser beam 110 on the skin tissue 108 falls within certain desired range. The laser apparatus 100 further comprises a control panel 112 for controlling the parameters of the laser source, such as output power, mode of operation (pulsed or continuous), pulse width, duty cycle, repetition rate, etc. Safety features of the laser apparatus 100 include a key switch 114 and an emergency switch 116, which may be used to turn off the laser source in case the intensity of the laser beam 110 exceeds certain safety level. The optical fiber 102 can be wound around a wheel 118 on the laser apparatus 100 to save space and protect the optical fiber 102 from mechanical damage.

The laser treatment may be applied to the skin tissue 108 before, during or after the Botox injection process. The laser beam 110 produces several therapeutic effects. The primary effect is the stimulation of cellular ATP production. The secondary mechanisms include effect on blood circulation, effect on stimulatory and regulatory mechanisms and effects on the immune system. As a result, the laser treatment triggers the activation of macrophages, induces increased procollagen synthesis, and/or increases the number of mast cells, which are beneficial for wound healing. The laser treatment also increases the serotonin level in the blood, which causes acceleration of the inflammatory process. In addition, the laser treatment increases the nerve cell action potential, which is helpful for pain relief. Through above and other identified therapeutic effects, the pain and inflammation reaction caused by the Botox injection can be reduced or eliminated.

A slight variation of the optical wand 106 is illustrated in FIG. 2. Here the top section of the optical wand 106 comprises a chamber 122 with a rolling massage ball 124 mounted therein. The massage ball 124 can rotate freely in the chamber 122 with controlled friction. The laser light is delivered onto the massage ball 124 through an optical fiber 102. The massage ball 124 is transparent at the wavelength of the laser light. Thus the laser light will transmit through the massage ball 124 to the skin tissue for treatment purposes. When a force is applied onto the optical wand 106, the massage ball 124 will roll on the skin tissue and deliver a pressure on the skin tissue that is in contact with the massage ball 124. On one hand, this rolling and kneading action causes an increase in blood circulation and fluid mobilization of the subcutaneous tissue for the relief of inflammation and pain. On the other hand, the action also causes a reduction in skin thickness and an increase in skin density. This change in skin tissue property helps to reduce the overall absorption and scattering loss of the laser light and allows the laser light to penetrate deeper into the skin and induce stronger therapeutic effects. The photo bio-modulation in turn helps to enhance the effect of the mechanical massage by stimulating inter or intra cellular response, increasing micro-circulation, etc. In yet another variation of the present embodiment, the massage ball (or simply an optical window with curved surface) 124 is rigidly affixed to the chamber 122 and moves together or in unison with the optical wand 106. In this variation, the mechanical massage is performed by kneading the skin tissue with the massage ball 124 without the rolling action.

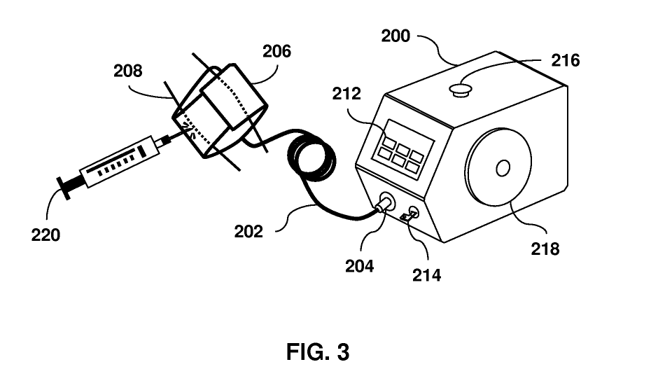

In another exemplary embodiment of the present invention as shown in FIG. 3, the laser light is delivered through a flexible light emitting pad or bandage 206, which can be wrapped over the skin tissue 208 to be treated with medical or cosmetic injections. The light emitting pad or bandage 206 may be in the form of woven fiber fabrics, side emitting fibers, or plastic plates as disclosed in U.S. Pat. Nos. 5,568,964 and 6,755,547. Referring to FIG. 2, the laser light produced by the laser apparatus 200 is first coupled into an optical fiber 202 at the output port 204 and then delivered into the light emitting pad 206, where the light emits from the side of the pad through refractive or diffusive effects. The light emitting pad 206 can be wrapped over the skin tissue before and/or after the injection process so that laser therapy can be applied to relieve pain and inflammation caused by the injection. Similar to that of FIG. 1, the laser apparatus 200 may comprise a control panel 212, a fiber wheel 218, as well as a key switch 214 and an emergency switch 116 as safety features.

In other variations of the present invention, the laser light can be delivered through free space optics instead of using optical fibers. The laser therapy procedure may be applied to other types of medical or cosmetic injections. For example, the present invention may be used in dermal filling, mesotherapy, or immunization shots, etc. for the reduction of pain and inflammation.

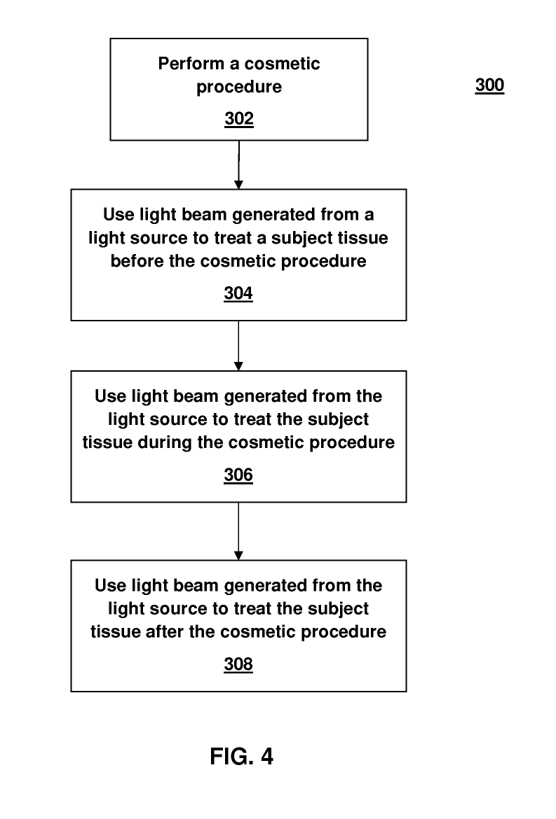

Referring to FIG. 4, a flowchart 300 of the present invention is shown. The process entails the performance of a cosmetic procedure that cause pain or discomfort to skin surface or tissues proximate to the skin surface (Step 302). The cosmetic procedure causes the skin of a subject to be penetrated. The cosmetic procedure may be Botox injection procedure or the like such as the procedures described supra. Use light beam generated from the light source to treat a subject tissue before the cosmetic procedure to reduce pain or other types of discomfort to a subject person (Step 304). Treat a subject tissue during the cosmetic procedure using the light beam generated from the light source (Step 306). Treat a subject tissue after the cosmetic procedure using the light beam generated from the light source (Step 308).

In the foregoing specification, specific embodiments of the present invention have been described. However, one of ordinary skill in the art appreciates that various modifications and changes can be made without departing from the scope of the present invention as set forth in the claims below. For example, the laser light source in the above disclosed embodiments may be replaced with a light emitting diode (LED) light source. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. A method for reducing pain and inflammation of a biological tissue caused by medical or cosmetic injections, the method comprising the steps of:
providing a light source to produce a light beam; and
causing the light beam to interact with the biological tissue before, during or after an injection process to reduce pain and inflammation of the tissue.

2. The method of claim 1, further comprising the step of providing an optical wand for delivering the light beam, wherein the optical wand comprises a massage component capable of providing mechanical massage to the biological tissue, and wherein the light beam is delivered through the massage component.

3. The method of claim 1, further comprising the step of providing a light emitting pad or bandage disposed to be wrapped over the biological tissue for delivering the light beam.

4. The method of claim 1, wherein the light source comprises a laser.

5. The method of claim 1, wherein the light source comprises a light emitting diode (LED).

6. The method of claim 1, wherein the wavelength of the light source ranges from ultraviolet, visible to infrared.

7. The method of claim 1, wherein the injection relates to Botox injection.

8. The method of claim 1, wherein the injection relates to dermal filling.

9. The method of claim 1, wherein the injection relates to mesotherapy.

10. The method of claim 1, wherein the injection relates to immunization shots.

11. An apparatus for reducing pain and inflammation of a biological tissue caused by medical or cosmetic injections, comprising:
a light source to produce a light beam, wherein the light beam interacts with the biological tissue before, during or after an injection process to reduce pain and inflammation of the tissue.

12. The apparatus of claim 11, further comprising an optical wand for delivering the light beam, wherein the optical wand comprises a massage component capable of providing mechanical massage to the biological tissue, and wherein the light beam is delivered through the massage component.

13. The apparatus of claim 11, further comprising a light emitting pad or bandage disposed to be wrapped over the biological tissue for delivering the light beam.

14. The apparatus of claim 11, wherein the light source comprises a laser.

15. The apparatus of claim 11, wherein the light source comprises a light emitting diode (LED).

16. The apparatus of claim 11, wherein the wavelength of the light source ranges from ultraviolet, visible to infrared.

17. The apparatus of claim 11, wherein the injection relates to Botox injection.

18. The apparatus of claim 11, wherein the injection relates to dermal filling.

19. The apparatus of claim 11, wherein the injection relates to mesotherapy.

20. The apparatus of claim 11, wherein the injection relates to immunization shots.
